(19) United States
(12) Patent Application Publication
(10) Pub. No.: US 2002/0049305 A1
(43) Pub. Date: Apr. 25, 2002

(54) Nucleotide sequences which code for the metF gene

(75) Inventors: Brigitte Bathe, Salzkotten (DE); Bettina Moeckel, Duesseldorf (DE); Walter Pfefferle, Halle (DE); Klaus Huthmacher, Gelnhausen (DE); Michael Binder, Steinhagen (DE); Dieter Greissinger, Niddatal (DE); Georg Thierbach, Bielefeld (DE)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: DEGUSSA AG, Duesseldorf (DE)

(21) Appl. No.: 09/919,935

(22) Filed: Aug. 2, 2001

Related U.S. Application Data
(60) Provisional application No. 60/294,279, filed on May 31, 2001.

(30) Foreign Application Priority Data
Aug. 2, 2000 (DE) 100 53 942.4
Feb. 28, 2001 (DE) 101 09 686.0

(51) Int. Cl. C07K 14/435; C07H 21/04; C12P 21/02; C12N 5/06

(52) U.S. Cl. 530/350; 536/23.5; 435/325; 435/320.1; 435/69.1

(57) ABSTRACT

An isolated polynucleotide comprising a polynucleotide sequence selected from the group consisting of

a) polynucleotide which is at least 70% identical to a polynucleotide that codes for a polypeptide which comprises the amino acid sequence of SEQ ID No. 2,

b) polynucleotide which codes for a polypeptide that comprises an amino acid sequence which is at least 70% identical to the amino acid sequence of SEQ ID No. 2,

c) polynucleotide which is complementary to the polynucleotides of a) or b), and

d) polynucleotide comprising at least 15 successive nucleotides of the polynucleotide sequence of a), b) or c),

and processes for the fermentative preparation of L-amino acids using coryneform bacteria in which at least the metF gene is present in enhanced form, and the use of the polynucleotide sequences as hybridization probes.

### BACKGROUND OF THE INVENTION

1. Field of the Invention

The invention provides nucleotide sequences from coryneform bacteria which code for the metF gene and a process for the fermentative preparation of amino acids, in particular L-methionine, using bacteria in which the metF gene is enhanced.

2. Description of the Related Art

L-Amino acids, in particular L-methionine, are used in human medicine and in the pharmaceuticals industry, in the foodstuffs industry and very particularly in animal nutrition.

It is known that amino acids are prepared by fermentation from strains of coryneform bacteria, in particular Corynebacterium glutamicum. Because of their great importance, work is constantly being undertaken to improve the preparation process. Improvements to the process can relate to fermentation measures, such as stirring and supply of oxygen, or to the composition of the nutrient media, such as the sugar concentration during the fermentation, or to the working up of the product by, for example, ion exchange chromatography, or to the intrinsic output properties of the microorganism itself.

Methods of mutagenesis, selection and mutant selection are used to improve the output properties of these microorganisms. Strains which are resistant to antimetabolites, such as e.g. the methionine analogue α-methyl-methionine, ethionine, norleucine, N-acetylnorleucine, S-trifluoromethylhomocysteine, 2-amino-5-heprenoitic acid, seleno-methionine, methionine-sulfoximine, methoxine, 1-aminocyclopentane-carboxylic acid, or are auxotrophic for metabolites of regulatory importance and produce amino acid, such as e.g. L-methionine, are obtained in this manner.

Recombinant DNA techniques have also been employed for some years for improving the Corynebacterium strains which produce L-amino acid, by amplifying individual amino acid biosynthesis genes and investigating their effect on amino acid production.

### SUMMARY OF THE INVENTION

An object of the present invention is to provide new measures for improved fermentative preparation of amino acids, in particular L-methionine.

When L-methionine or methionine are mentioned in the following, the salts, such as methionine hydrochloride or methionine sulfate are also meant.

The invention provides an isolated polynucleotide from coryneform bacteria, comprising a polynucleotide sequence which codes for the metF gene, chosen from the group consisting of

a) polynucleotide which is at least 70% identical to a polynucleotide that codes for a polypeptide which comprises the amino acid sequence of SEQ ID No. 2,

b) polynucleotide which codes for a polypeptide that comprises an amino acid sequence which is at least 70% identical to the amino acid sequence of SEQ ID No. 2,

c) polynucleotide which is complementary to the polynucleotides of a) or b), and

d) polynucleotide comprising at least 15 successive nucleotides of the polynucleotide sequence of a), b) or c),

and the corresponding polypeptide preferably having the enzymatic activity of methylene tetrahydrofolate reductase.

The invention also provides the above-mentioned polynucleotides as DNA which is capable of replication, comprising:

(i) the nucleotide sequence shown in SEQ ID No. 1, or

(ii) at least one sequence which corresponds to sequence (i) within the range of the degeneration of the genetic code, or

(iii) at least one sequence which hybridizes with the sequence complementary to sequence (i) or (ii), and optionally

(iv) sense mutations of neutral function in (i).

The invention also provides

a polynucleotide comprising the nucleotide sequence as shown in SEQ ID No. 1,

a polynucleotide that codes for a polypeptide which comprises the amino acid sequence as shown in SEQ ID No. 2,

a vector containing the polynucleotide according to the invention, in particular a shuttle vector or plasmid vector, and

and coryneform bacteria serving as the host cell, which contain the vector or in which the metF gene is enhanced.

The invention also provides polynucleotides which are obtained by screening a corresponding gene library, which comprises the complete gene having the polynucleotide sequence corresponding to SEQ ID No. 1, by means of hybridization with a probe which comprises the sequence of the polynucleotide mentioned, according to SEQ ID No. 1 or a fragment thereof, and isolation of the DNA sequence mentioned.

### BRIEF DESCRIPTION OF THE FIGURES

FIG. 1 shows plasmid pCREmetF.

### DETAILED DESCRIPTION OF THE PREFERRED EMBODIMENTS

Polynucleotides which comprise the sequences according to the invention are suitable as hybridization probes for RNA, cDNA and DNA, in order to isolate, in the full length, nucleic acids or polynucleotides or genes which code for methylene tetrahydrofolate reductase or to isolate those nucleic acids or polynucleotides or genes which have a high similarity of sequence or homology to methylene tetrahydrofolate reductase.

Polynucleotides according to the invention are furthermore suitable as primers with which the DNA of genes that code for methylene tetrahydrofolate reductase can be prepared by the polymerase chain reaction (PCR).

Such oligonucleotides that serve as probes or primers comprise at least 30, preferably at least 20, very particularly at least 15 successive nucleotides. Oligonucleotides which have a length of at least 40 or 50 nucleotides are also suitable. Oligonucleotides with a length of at least 100, 150, 200, 250 or 300 nucleotides are optionally also suitable.

“Isolated” means separated out of its natural environment.

“Polynucleotide” in general relates to polyribonucleotides and polydeoxyribonucleotides, it being possible for these to be non-modified RNA or DNA or modified RNA or DNA.

“Polypeptides” are understood as meaning peptides or proteins which comprise two or more amino acids bonded via peptide bonds.

The polypeptides according to the invention include a polypeptide according to SEQ ID No. 2, in particular those with the biological activity of methylene tetrahydrofolate reductase, and also those which are at least 70%, preferably at least 80% and in particular which are at least 90% to 95% identical to the polypeptide according to SEQ ID No. 2 and have the activity mentioned.

The invention moreover provides a process for the fermentative preparation of amino acids, in particular L-methionine, using coryneform bacteria which in particular already produce amino acids, and in which the nucleotide sequences which code for the metF gene are enhanced, in particular over-expressed.

The term “enhancement” in this connection describes an increase in the intracellular activity of one or more enzymes in a microorganism which are coded by the corresponding DNA, for example by increasing the number of copies of the gene or genes, using a potent promoter or using a gene which codes for a corresponding enzyme having a high activity, and optionally combining these measures.

By enhancement measures, in particular over-expression, the activity or concentration of the corresponding protein is in general increased by at least 10%, 25%, 50%, 75%, 100%, 150%, 200%, 300%, 400% or 500%, up to a maximum of 1000% or 2000%, based on the starting microorganism.

The microorganisms which the present invention provides can prepare L-amino acids, in particular L-methionine, from glucose, sucrose, lactose, fructose, maltose, molasses, starch, cellulose or from glycerol and ethanol. They can be representatives of coryneform bacteria, in particular of the genus Corynebacterium. Of the genus Corynebacterium, there may be mentioned in particular the species Corynebacterium glutamicum, which is known among experts for its ability to produce L-amino acids.

Suitable strains of the genus Corynebacterium, in particular of the species Corynebacterium glutamicum (C. glutamicum), are in particular the known wild-type strains

Corynebacterium glutamicum ATCC13032

Corynebacterium acetoglutamicum ATCC15806

Corynebacterium acetoacidophilum ATCC13870

Corynebacterium thermoaminogenes FERM BP-1539

Corynebacterium melassecola ATCC17965

Brevibacterium flavum ATCC14067

Brevibacterium lactofermentum ATCC13869 and

Brevibacterium divaricatum ATCC14020

or L-amino acid-producing mutants or strains prepared therefrom, such as, for example, the L-methionine-producing strain

Corynebacterium glutamicum ATCC21608.

The new metF gene from C. glutamicum which codes for the enzyme methylene tetrahydrofolate reductase [EC:1.7.99.5] has been isolated.

To isolate the metF gene or also other genes of C. glutamicum, a gene library of this microorganism is first set up in Escherichia coli (E. coli). The setting up of gene libraries is described in generally known textbooks and handbooks. The textbook by Winnacker: Gene und Klone, Eine Einführung in die Gentechnologie (Verlag Chemie, Weinheim, Germany, 1990), or the handbook by Sambrook et al.: Molecular Cloning, A Laboratory Manual (Cold Spring Harbor Laboratory Press, 1989) may be mentioned as an example. A well-known gene library is that of the E. coli K-12 strain W3110 set up in λ vectors by Kohara et al. (Cell 50, 495 -508 (1987)). Bathe et al. (Molecular and General Genetics, 252:255-265, 1996) describe a gene library of C. glutamicum ATCC13032, which was set up with the aid of the cosmid vector SuperCos I (Wahl et al., 1987, Proceedings of the National Academy of Sciences USA, 84:2160-2164) in the E. coli K-12 strain NM554 (Raleigh et al., 1988, Nucleic Acids Research 16:1563-1575).

Börmann et al. (Molecular Microbiology 6(3), 317-326) (1992)) in turn describe a gene library of C. glutamicum ATCC13032 using the cosmid pHC79 (Hohn and Collins, Gene 11, 291-298 (1980)).

To prepare a gene library of C. glutamicum in E. coli it is also possible to use plasmids such as pBR322 (Bolivar, Life Sciences, 25, 807-818 (1979)) or pUC9 (Vieira et al., 1982, Gene, 19:259-268). Suitable hosts are, in particular, those E. coli strains which are restriction- and recombination-defective. An example of these is the strain DH5αmcr, which has been described by Grant et al. (Proceedings of the National Academy of Sciences USA, 87 (1990) 4645-4649). The long DNA fragments cloned with the aid of cosmids can in turn be subcloned in the usual vectors suitable for sequencing and then sequenced, as is described e.g. by Sanger et al. (Proceedings of the National Academy of Sciences of the United States of America, 74:5463-5467, 1977).

The resulting DNA sequences can then be investigated with known algorithms or sequence analysis programs, such as that of Staden (Nucleic Acids Research 14, 217-232(1986)), that of Marck (Nucleic Acids Research 16, 1829-1836 (1988)) or the GCG program of Butler (Methods of Biochemical Analysis 39, 74-97 (1998)).

The new DNA sequence of C. glutamicum which codes for the metF gene and which, as SEQ ID No. 1, is a constituent of the present invention has been found. The amino acid sequence of the corresponding protein has furthermore been derived from the present DNA sequence by the methods described above. The resulting amino acid sequence of the metF gene product is shown in SEQ ID No. 2.

Coding DNA sequences which result from SEQ ID No. 1 by the degeneracy of the genetic code are also a constituent of the invention. In the same way, DNA sequences which hybridize with SEQ ID No. 1 or parts of SEQ ID No. 1 are a constituent of the invention. Conservative amino acid exchanges, such as e.g. exchange of glycine for alanine or of aspartic acid for glutamic acid in proteins, are furthermore known among experts as “sense mutations” which do not lead to a fundamental change in the activity of the protein, i.e. they are of neutral function.

It is furthermore known that changes at the N and/or C terminus of a protein must not substantially impair and may even stabilize the function thereof. Information in this context can be found in Ben-Bassat et al. (Journal of Bacteriology 169:751-757 (1987)), in O'Regan et al. (Gene 77:237-251 (1989)), in Sahin-Toth et al. (Protein Sciences 3:240-247 (1994)), in Hochuli et al. (Bio/Technology 6:1321-1325 (1988)) and in known textbooks of genetics and molecular biology. Amino acid sequences which result in a corresponding manner from SEQ ID No. 2 are also a constituent of the invention.

In the same way, DNA sequences which hybridize with SEQ ID No. 1 or parts of SEQ ID No. 1 are a constituent of the invention. Finally, DNA sequences which are prepared by the polymerase chain reaction (PCR) using primers which result from SEQ ID No. 1 are a constituent of the invention. Such oligonucleotides typically have a length of at least 15 nucleotides.

Instructions for identifying DNA sequences by means of hybridization can be found in the handbook “The DIG System Users Guide for Filter Hybridization” from Boehringer Mannheim GmbH (Mannheim, Germany, 1993) and in Liebl et al. (International Journal of Systematic Bacteriology (1991) 41: 255-260). Instructions for amplification of DNA sequences with the aid of the polymerase chain reaction (PCR) can be found in the handbook by Gait: Oligonucleotide Synthesis: A Practical Approach (IRL Press, Oxford, UK, 1984) and in Newton and Graham: PCR (Spektrum Akademischer Verlag, Heidelberg, Germany, 1994).

It has been found that coryneform bacteria produce amino acids, in particular L-methionine, in an improved manner after over-expression of the metF gene.

To achieve an over-expression, the number of copies of the corresponding genes can be increased, or the promoter and regulation region or the ribosome binding site upstream of the structural gene can be mutated. Expression cassettes which are incorporated upstream of the structural gene act in the same way. By inducible promoters, it is additionally possible to increase the expression in the course of fermentative L-methionine production. The expression is likewise improved by measures to prolong the life of the m-RNA. Furthermore, the enzyme activity is also increased by preventing the degradation of the enzyme protein. The genes or gene constructs can either be present in plasmids with a varying number of copies, or can be integrated and amplified in the chromosome. Alternatively, an over-expression of the genes in question can furthermore be achieved by changing the composition of the media and the culture procedure.

Instructions in this context can be found in Martin et al. (Bio/Technology 5, 137-146 (1987)), in Guerrero et al. (Gene 138, 35-41 (1994)), Tsuchiya and Morinaga (Bio/Technology 6, 428-430 (1988)), in Eikmanns et al. (Gene 102, 93-98 (1991)), in European Patent Specification 0 472 869, in U.S. Pat. No. 4,601,893, in Schwarzer and Pühler (Bio/Technology 9, 84-87 (1991), in Reinscheid et al. (Applied and Environmental Microbiology 60, 126-132 (1994)), in LaBarre et al. (Journal of Bacteriology 175, 1001-1007 (1993)), in Patent Application WO96/15246, in Malumbres et al. (Gene 134, 15 -24 (1993)), in Japanese Laid-Open Specification JP-A-10-229891, in Jensen and Hammer (Biotechnology and Bioengineering 58, 191-195 (1998)), in Makrides (Microbiological Reviews 60:512-538 (1996)) and in known textbooks of genetics and molecular biology.

By way of example, for enhancement the metF gene according to the invention was over-expressed with the aid of episomal plasmids. Suitable plasmids are those which are replicated in coryneform bacteria. Numerous known plasmid vectors, such as e.g. pZ1 (Menkel et al., Applied and Environmental Microbiology (1989) 64: 549-554), pEKEx1 (Eikmanns et al., Gene 102:93-98 (1991)) or pHS2-1 (Sonnen et al., Gene 107:69-74 (1991)) are based on the cryptic plasmids pHM1519, pBL1 or pGA1. Other plasmid vectors, such as those based on pCG4 (U.S. Pat. No. 4,489,160), or pNG2 (Serwold-Davis et al., FEMS Microbiology Letters 66, 119-124 (1990)), or pAG1 (U.S. Pat. No. 5,158,891), can be used in the same manner.

Plasmid vectors which are furthermore suitable are also those with the aid of which the process of gene amplification by integration into the chromosome can be used, as has been described, for example, by Reinscheid et al. (Applied and Environmental Microbiology 60, 126-132 (1994)) for duplication or amplification of the hom-thrB operon. In this method, the complete gene is cloned in a plasmid vector which can replicate in a host (typically E. coli), but not in C. glutamicum. Possible vectors are, for example, pSUP301 (Simon et al., Bio/Technology 1, 784-791 (1983)), pK18mob or pK19mob (Schäfer et al., Gene 145, 69-73 (1994)), PGEM-T (Promega corporation, Madison, Wis., USA), pCR2.1-TOPO (Shuman (1994). Journal of Biological Chemistry 269:32678-84; U.S. Pat. No. 5,487,993), pCR®Blunt (Invitrogen, Groningen, Holland; Bernard et al., Journal of Molecular Biology, 234: 534-541 (1993)), pEM1 (Schrumpf et al, 1991, Journal of Bacteriology 173:4510-4516) or pBGS8 (Spratt et al.,1986, Gene 41: 337-342). The plasmid vector which contains the gene to be amplified is then transferred into the desired strain of C. glutamicum by conjugation or transformation. The method of conjugation is described, for example, by Schafer et al. (Applied and Environmental Microbiology 60, 756-759 (1994)). Methods for transformation are described, for example, by Thierbach et al. (Applied Microbiology and Biotechnology 29, 356-362 (1988)), Dunican and Shivnan (Bio/Technology 7, 1067-1070 (1989)) and Tauch et al. (FEMS Microbiological Letters 123, 343-347 (1994)). After homologous recombination by means of a “cross over” event, the resulting strain contains at least two copies of the gene in question.

In addition, it may be advantageous for the production of amino acids, in particular L-methionine, to enhance one or more enzymes of the particular biosynthesis pathway, of glycolysis, of anaplerosis, of the citric acid cycle or of amino acid export, in addition to the metF gene.

Thus for the preparation of amino acids, in particular L-methionine, one or more genes chosen from the group consisting of

the gap gene which codes for glyceraldehyde 3-phosphate dehydrogenase (Eikmanns (1992), Journal of Bacteriology 174:6076-6086),

the tpi gene which codes for triose phosphate isomerase (Eikmanns (1992), Journal of Bacteriology 174:6076-6086),

the pgk gene which codes for 3-phosphoglycerate kinase (Eikmanns (1992), Journal of Bacteriology 174:6076-6086),

the pyc gene which codes for pyruvate carboxylase (Eikmanns (1992), Journal of Bacteriology 174:6076-6086),

the lysC gene which codes for a feed-back resistant aspartate kinase (ACCESSION NUMBER P26512 ; EP-B-0387527; EP-A-0699759),

the metA gene which codes for homoserine O-acetyltransferase (ACCESSION Number AF052652),

the metB gene which codes for cystathionine gamma-synthase (ACCESSION Number AF126953),

the aecD gene which codes for cystathionine gamma-lyase (ACCESSION Number M89931)

the glyA gene which codes for serine hydroxymethyltransferase (JP-A-08107788),

the metY gene which codes for O-acetylhomoserine sulfhydrylase (DSM 13556)

can be enhanced, in particular over-expressed.

It may furthermore be advantageous for the production of amino acids, in particular L-methionine, in addition to the enhancement of the metF gene, for one or more genes chosen from the group consisting of

the thrB gene which codes for homoserine kinase (ACCESSION Number P08210),

the ilvA gene which codes for threonine dehydratase (ACCESSION Number Q04513),

the thrC gene which codes for threonine synthase (ACCESSION Number P23669),

the ddh gene which codes for meso-diaminopimelate D-dehydrogenase (ACCESSION Number Y00151),

the pck gene which codes for phosphoenol pyruvate carboxykinase (DE 199 50 409.1; DSM 13047),

the pgi gene which codes for glucose 6-phosphate isomerase (US 09/396,478; DSM 12969),

the poxB gene which codes for pyruvate oxidase (DE: 1995 1975.7; DSM 13114)

to be attenuated, in particular for the expression thereof to be reduced.

The term “attenuation” in this connection describes the reduction or elimination of the intracellular activity of one or more enzymes (proteins) in a microorganism which are coded by the corresponding DNA, for example by using a weak promoter or using a gene or allele which codes for a corresponding enzyme with a low activity or inactivates the corresponding gene or enzyme (protein), and optionally combining these measures.

By attenuation measures, the activity or concentration of the corresponding protein is in general reduced to 0 to 50%, 0 to 25%, 0 to 10% or 0 to 5% of the activity or concentration of the wild-type protein.

In addition to over-expression of the metF gene it may furthermore be advantageous for the production of amino acids, in particular L-methionine, to eliminate undesirable side reactions, (Nakayama: “Breeding of Amino Acid Producing Micro-organisms”, in: Overproduction of Microbial Products, Krumphanzl, Sikyta, Vanek (eds.), Academic Press, London, UK, 1982).

The microorganisms prepared according to the invention can be cultured continuously or discontinuously in the batch process (batch culture) or in the fed batch (feed process) or repeated fed batch process (repetitive feed process) for the purpose of production of amino acids, in particular L-methionine. A summary of known culture methods is described in the textbook by Chmiel (Bioprozesstechnik 1. Einführung in die Bioverfahrenstechnik (Gustav Fischer Verlag, Stuttgart, 1991)) or in the textbook by Storhas (Bioreaktoren und periphere Einrichtungen (Vieweg Verlag, Braunschweig/Wiesbaden, 1994)).

The culture medium to be used must meet the requirements of the particular strains in a suitable manner. Descriptions of i5 culture media for various microorganisms are contained in the handbook “Manual of Methods for General Bacteriology” of the American Society for Bacteriology (Washington D.C., USA, 1981).

Sugars and carbohydrates, such as e.g. glucose, sucrose, lactose, fructose, maltose, molasses, starch and cellulose, oils and fats, such as e.g. soya oil, sunflower oil, groundnut oil and coconut fat, fatty acids, such as e.g. palmitic acid, stearic acid and linoleic acid, alcohols, such as e.g. glycerol and ethanol, and organic acids, such as e.g. acetic acid, can be used as the source of carbon. These substance can be used individually or as a mixture.

Organic nitrogen-containing compounds, such as peptones, yeast extract, meat extract, malt extract, corn steep liquor, soya bean flour and urea, or inorganic compounds, such as ammonium sulfate, ammonium chloride, ammonium phosphate, ammonium carbonate and ammonium nitrate, can be used as the source of nitrogen. The sources of nitrogen can be used individually or as a mixture.

Organic and inorganic sulfur-containing compounds, such as, for example, sulfides, sulfites, sulfates and thiosulfates, can be used as a source of sulfur, in particular for the preparation of methionine.

Phosphoric acid, potassium dihydrogen phosphate or dipotassium hydrogen phosphate or the corresponding sodium-containing salts can be used as the source of phosphorus. The culture medium must furthermore comprise salts of metals, such as e. g. magnesium sulfate or iron sulfate, which are necessary for growth. Finally, essential growth substances, such as amino acids and vitamins, can be employed in addition to the above-mentioned substances. Suitable precursors can moreover be added to the culture medium. The starting substances mentioned can be added to the culture in the form of a single batch, or can be fed in during the culture in a suitable manner.

Basic compounds, such as sodium hydroxide, potassium hydroxide, ammonia or aqueous ammonia, or acid compounds, such as phosphoric acid or sulfuric acid, can be employed in a suitable manner to control the pH of the culture. Antifoams, such as e.g. fatty acid polyglycol esters, can be employed to control the development of foam. Suitable substances having a selective action, such as e.g. antibiotics, can be added to the medium to maintain the stability of plasmids. To maintain aerobic conditions, oxygen or oxygen-containing gas mixtures, such as e.g. air, are introduced into the culture. The temperature of the culture is usually 20° C. to 45° C., and preferably 25° C. to 40° C. Culturing is continued until a maximum of the desired product has formed. This target is usually reached within 10 hours to 160 hours.

The fermentation broths obtained in this way, in particular containing L-methionine, usually have a dry weight of 7.5 to 25 wt. % and contain L-methionine. It is furthermore also advantageous if the fermentation is conducted in a sugar-limited procedure at least at the end, but in particular over at least 30% of the duration of the fermentation. That is to say, the concentration of utilizable sugar in the fermentation medium is reduced to ≧0 to 3 g/l during this period.

The fermentation broth prepared in this manner, in particular containing L-methionine, is then further processed. Depending on requirements all or some of the biomass can be removed from the fermentation broth by separation methods, such as centrifugation, filtration, decanting or a combination thereof, or it can be left completely in. This broth is then thickened or concentrated by known methods, such as with the aid of a rotary evaporator, thin film evaporator, falling film evaporator, by reverse osmosis, or by nanofiltration. This concentrated fermentation broth can then be worked up by methods of freeze drying, spray drying, spray granulation or by other processes to give a preferably free-flowing, finely divided powder.

This free-flowing, finely divided powder can then in turn by converted by suitable compacting or granulating processes into a coarse-grained, readily free-flowing, storable and largely dust-free product. In the granulation or compacting it is advantageous to employ conventional organic or inorganic auxiliary substances or carriers, such as starch, gelatin, cellulose derivatives or similar substances, such as are conventionally used as binders, gelling agents or thickeners in foodstuffs or feedstuffs processing, or further substances, such as, for example, silicas, silicates or stearates.

“Free-flowing” is understood as meaning powders which flow unimpeded out of the vessel with the opening of 5 mm (millimeters) of a series of glass outflow vessels with outflow openings of various sizes (Klein, Seifen, Öle, Fette, Wachse 94, 12 (1968)).

As described here, “finely divided” means a powder with a predominant content (>50%) having a particle size of 20 to 200 μm diameter. “Coarse-grained” means products with a predominant content (>50%) having a particle size of 200 to 2000 μm diameter. In this context, “dust-free” means that the product contains only small contents (<5%) having particle sizes of less than 20 μm diameter. The particle size determination can be carried out with methods of laser diffraction spectrometry. The corresponding methods are described in the textbook on “Teilchengröβenmessung in der Laborpraxis” by R. H. Müller and R. Schuhmann, Wissenschaftliche Verlagsgesellschaft Stuttgart (1996) or in the textbook “Introduction to Particle Technology” by M. Rhodes, Verlag Wiley & Sons (1998).

“Storable” in the context of this invention means a product which can be stored for up to 120 days, preferably up to 52 weeks, particularly preferably 60 months, without a substantial loss (<5%) of methionine occurring.

Alternatively, however, the product can be absorbed on to an organic or inorganic carrier substance which is known and conventional in feedstuffs processing, for example, silicas, silicates, grits, brans, meals, starches, sugars or others, and/or mixed and stabilized with conventional thickeners or binders. Use examples and processes in this context are described in the literature (Die Mühle+Mischfuttertechnik 132 (1995) 49, page 817).

Finally, the product can be brought into a state in which it is stable to digestion by animal stomachs, in particular the stomach of ruminants, by coating processes (“coating”) using film-forming agents, such as, for example, metal carbonates, silicas, silicates, alginates, stearates, starches, gums and cellulose ethers, as described in DE C 4100920.

If the biomass is separated off during the process, further inorganic solids, for example added during the fermentation, are in general removed. In addition, the animal feedstuffs additive according to the invention comprises at least the predominant proportion of the further substances, in particular organic substances, which are formed or added and are present in solution in the fermentation broth, where these have not been separated off by suitable processes.

In one aspect of the invention, the biomass can be separated off to the extent of up to 70%, preferably up to 80%, preferably up to 90%, preferably up to 95%, and particularly preferably up to 100%. In another aspect of the invention, up to 20% of the biomass, preferably up to 15%, preferably up to 10%, preferably up to 5%, particularly preferably no biomass is separated off.

These organic substances include organic by-products which are optionally produced, in addition to the L-methionine, and optionally discharged by the microorganisms employed in the fermentation. These include L-amino acids chosen from the group consisting of L-lysine, L-valine, L-threonine, L-alanine or L-tryptophan. They include vitamins chosen from the group consisting of vitamin B1 (thiamine), vitamin B2 (riboflavin),vitamin B5 (pantothenic acid), vitamin B6 (pyridoxine), vitamin B12 (cyanocobalamin), nicotinic acid/nicotinamide and vitamin E (tocopherol). They also include organic acids which carry one to three carboxyl groups, such as, for example, acetic acid, lactic acid, citric acid, malic acid or fumaric acid. Finally, they also include sugars, such as, for example, trehalose. These compounds are optionally desired if they improve the nutritional value of the product.

These organic substances, including L-methionine and/or D-methionine and/or the racemic mixture D,L-methionine, can also be added, depending on requirements, as a concentrate or pure substance in solid or liquid form during a suitable process step. These organic substances mentioned can be added individually or as mixtures to the resulting or concentrated fermentation broth, or also during the drying or granulation process. It is likewise possible to add an organic substance or a mixture of several organic substances to the fermentation broth and a further organic substance or a further mixture of several organic substances during a later process step, for example granulation.

The product described above is suitable as a feedstuffs additive, i.e. feed additive, for animal nutrition.

The L-methionine content of the animal feedstuffs additive is conventionally 1 wt. % to 80 wt. %, preferably 2 wt. % to 80 wt. %, particularly preferably 4 wt. % to 80 wt. %, and very particularly preferably 8 wt. % to 80 wt. %, based on the dry weight of the animal feedstuffs additive. Contents of 1 wt. % to 60 wt. %, 2 wt. % to 60 wt. %, 4 wt. % to 60 wt. %, 6 wt. % to 60 wt. %, 1 wt. % to 40 wt. %, 2 wt. % to 40 wt. % or 4 wt. % to 40 wt. % are likewise possible. The water content of the feedstuffs additive is conventionally up to 5 wt. %, preferably up to 4 wt. %, and particularly preferably less than 2 wt. %.

The invention also provides a process for the preparation of an L-methionine-containing animal feedstuffs additive from fermentation broths, which comprises the steps

a) culture and fermentation of an L-methionine-producing microorganism in a fermentation medium;

b) removal of water from the L-methionine-containing fermentation broth (concentration);

c) removal of an amount of 0 to 100 wt. % of the biomass formed during the fermentation; and

d) drying of the fermentation broth obtained according to a) and/or b) to obtain the animal feedstuffs additive in the desired powder or granule form.

If desired, one or more of the following steps can furthermore be carried out in the process according to the invention:

e) addition of one or more organic substances, including L-methionine and/or D-methionine and/or the racemic mixture D,L-methionine, to the products obtained according to a), b) and/or c);

f) addition of auxiliary substances chosen from the group consisting of silicas, silicates, stearates, grits and bran to the substances obtained according to a) to d) for stabilization and to increase the storability; or

g) conversion of the substances obtained according to a) to e) into a form stable to the animal stomach, in particular rumen, by coating with film-forming agents.

The analysis of L-methionine can be carried out by ion exchange chromatography with subsequent ninhydrin derivation, as described by Spackman et al. (Analytical Chemistry, 30, (1958), 1190).

The process according to the invention is used for the fermentative preparation of amino acids, in particular L-methionine.

The present invention is explained in more detail in the following with the aid of embodiment examples.

### Example 1

### Preparation of a genomic cosmid gene library from Corynebacterium glutamicum ATCC 13032

Chromosomal DNA from Corynebacterium glutamicum ATCC 13032 was isolated as described by Tauch et al. (1995, Plasmid 33:168-179) and partly cleaved with the restriction enzyme Sau3AI (Amersham Pharmacia, Freiburg, Germany, Product Description Sau3AI, Code no. 27-0913-02). The DNA fragments were dephosphorylated with shrimp alkaline phosphatase (Roche Diagnostics GmbH, Mannheim, Germany, Product Description SAP, Code no. 1758250). The DNA of the cosmid vector SuperCos1 (Wahl et al. (1987) Proceedings of the National Academy of Sciences USA 84:2160-2164), obtained from Stratagene (La Jolla, USA, Product Description SuperCosl Cosmid Vector Kit, Code no. 251301) was cleaved with the restriction enzyme XbaI (Amersham Pharmacia, Freiburg, Germany, Product Description XbaI, Code no. 27-0948-02) and likewise dephosphorylated with shrimp alkaline phosphatase.

The cosmid DNA was then cleaved with the restriction enzyme BamHI (Amersham Pharmacia, Freiburg, Germany, Product Description BamHI, Code no. 27-0868-04). The cosmid DNA treated in this manner was mixed with the treated ATCC13032 DNA and the batch was treated with T4 DNA ligase (Amersham Pharmacia, Freiburg, Germany, Product Description T4-DNA-Ligase, Code no. 27-0870-04). The ligation mixture was then packed in phages with the aid of Gigapack II XL Packing Extract (Stratagene, La Jolla, USA, Product Description Gigapack II XL Packing Extract, Code no. 200217).

For infection of the E. coli strain NM554 (Raleigh et al. 1988, Nucleic Acid Research 16:1563-1575) the cells were taken up in 10 mM MgSO₄ and mixed with an aliquot of the phage suspension. The infection and titering of the cosmid library were carried out as described by Sambrook et al. (1989, Molecular Cloning: A laboratory Manual, Cold Spring Harbor), the cells being plated out on LB agar (Lennox, 1955, Virology, 1:190) with 100 mg/l ampicillin. After incubation overnight at 37° C., recombinant individual clones were selected.

### Example 2

### Isolation and sequencing of the metF gene

The cosmid DNA of an individual colony was isolated with the Qiaprep Spin Miniprep Kit (Product No. 27106, Qiagen, Hilden, Germany) in accordance with the manufacturer's instructions and partly cleaved with the restriction enzyme Sau3AI (Amersham Pharmacia, Freiburg, Germany, Product Description Sau3AI, Product No. 27-0913-02). The DNA fragments were dephosphorylated with shrimp alkaline phosphatase (Roche Diagnostics GmbH, Mannheim, Germany, Product Description SAP, Product No. 1758250). After separation by gel electrophoresis, the cosmid fragments in the size range of 1500 to 2000 bp were isolated with the QiaExII Gel Extraction Kit (Product No. 20021, Qiagen, Hilden, Germany).

The DNA of the sequencing vector pZero-1, obtained from Invitrogen (Groningen, The Netherlands, Product Description Zero Background Cloning Kit, Product No. K2500-01) was cleaved with the restriction enzyme BamHI (Amersham Pharmacia, Freiburg, Germany, Product Description BamHI, Product No. 27-0868-04). The ligation of the cosmid fragments in the sequencing vector pZero-1 was carried out as described by Sambrook et al. (1989, Molecular Cloning: A Laboratory Manual, Cold Spring Harbor), the DNA mixture being incubated overnight with T4 ligase (Pharmacia Biotech, Freiburg, Germany). This ligation mixture was then electroporated (Tauch et al. 1994, FEMS Microbiol Letters, 123:343-7) into the E. coli strain DH5αMCR (Grant, 1990, Proceedings of the National Academy of Sciences U.S.A., 87:4645-4649) and plated out on LB agar (Lennox, 1955, Virology, 1:190) with 50 mg/l zeocin.

The plasmid preparation of the recombinant clones was carried out with Biorobot 9600 (Product No. 900200, Qiagen, Hilden, Germany). The sequencing was carried out by the dideoxy chain termination method of Sanger et al. (1977, Proceedings of the National Academy of Sciences U.S.A., 74:5463-5467) with modifications according to Zimmermann et al. (1990, Nucleic Acids Research, 18:1067). The “RR dRhodamin Terminator Cycle Sequencing Kit” from PE Applied Biosystems (Product No. 403044, Weiterstadt, Germany) was used. The separation by gel electrophoresis and analysis of the sequencing reaction were carried out in a “Rotiphoresis NF Acrylamide/Bisacrylamide” Gel (29:1) (Product No. A124.1, Roth, Karlsruhe, Germany) with the “ABI Prism 377” sequencer from PE Applied Biosystems (Weiterstadt, Germany).

The raw sequence data obtained were then processed using the Staden program package (1986, Nucleic Acids Research, 14:217-231) version 97-0. The individual sequences of the pZerol derivatives were assembled to a continuous contig. The computer-assisted coding region analysis was prepared with the XNIP program (Staden, 1986, Nucleic Acids Research, 14:217-231).

The resulting nucleotide sequence is shown in SEQ ID No. 1. Analysis of the nucleotide sequence showed an open reading frame of 1046 base pairs, which was called the metF gene. The metF gene codes for a protein of 349 amino acids.

### Example 3

### Preparation of the strain C. glutamicum ATCC13032/pCREmetF

3.1 Amplification of the metF gene

From the strain ATCC13032, chromosomal DNA was isolated by the method of Eikmanns et al. (Microbiology 140: 1817 -1828 (1994)). Starting from the nucleotide sequences of the methionine biosynthesis genes metF (SEQ ID No. 1) of C. glutamicum ATCC13032, the following oligonucleotides were chosen for the polymerase chain reaction (PCR) (see SEQ ID No. 3 and SEQ ID No. 4):

metF-EVP5:

5′-GATCTAGGATCCAAAGGAGGACAACCATGTCCCTAACGAACATCCC-3′

metF-EVP3:

5′-GATCTACTCGAGTTCTTCTAGTTGGCTCGGCA-3′

The primers shown were synthesized by MWG-Biotech AG (Ebersberg, Germany) and the PCR reaction was carried out by the standard PCR method of Innis et al. (PCR protocols. A guide to methods and applications, 1990, Academic Press) with Pwo-Polymerase from Roche Diagnostics GmbH (Mannheim, Germany). With the aid of the polymerase chain reaction, the primers allow amplification of a DNA fragment 792 bp in size, which carries the complete metF gene, which is suitable for expression.

Furthermore, the primer metF-EVP5 contains the sequence for the cleavage site of the restriction endonuclease BamHI and the primer metF-EVP3 the cleavage site of the restriction endonuclease XhoI, which are marked by underlining in the nucleotide sequence shown above.

The metF fragment 792 bp in size was cleaved with the restriction endonucleases BamHI and XhoI. The batch was separated by gel electrophoresis and the metF fragment was then isolated from the agarose gel with the QiaExII Gel Extraction Kit (Product No. 20021, Qiagen, Hilden, Germany).

3.2 Cloning of metF in the vector pZ8-1

The E. coli-C. glutamicum shuttle expression vector pZ8-1 (EP 0 375 889) was used as the base vector for the expression.

DNA of the plasmid pZ8-1 was cleaved completely with the restriction enzymes BamHI and SalI and then dephosphorylated with shrimp alkaline phosphatase (Roche Diagnostics GmbH, Mannheim, Germany, Product Description SAP, Product No. 1758250).

The metF fragment isolated from the agarose gel in example 3.1 and cleaved with the restriction endonucleases BamHI and XhoI was mixed with the vector pZ8-1 prepared in this way and the batch was treated with T4 DNA ligase (Amersham Pharmacia, Freiburg, Germany, Product Description T4-DNA-Ligase, Code no. 27-0870-04).

The ligation batch was transformed in the E. coli strain DH5αmcr (Hanahan, In: DNA cloning. A Practical Approach. Vol. I. IRL-Press, Oxford, Washington D.C., USA). Selection of plasmid-carrying cells was made by plating out the transformation batch on LB agar (Lennox, 1955, Virology, 1:190) with 50 mg/l kanamycin. After incubation overnight at 37° C., recombinant individual clones were selected. Plasmid DNA was isolated from a transformant with the Qiaprep Spin Miniprep Kit (Product No. 27106, Qiagen, Hilden, Germany) in accordance with the manufacturer's instructions and checked by restriction cleavage. The resulting plasmid was called pCREmetF.

3.3 Preparation of the strain C. glutamicum ATCC13032/pCREmetF

The vector pCREmetF obtained in example 3.2 was electroporated in the strain C. glutamicum ATCC13032 using the electroporation method described by Liebl et al. (FEMS Microbiology Letters, 53:299-303 (1989)). Selection of the plasmid-carrying cells took place on LBHIS agar comprising 18.5 g/l brain-heart infusion broth, 0.5 M sorbitol, 5 g/l Bacto-tryptone, 2.5 g/l Bacto-yeast extract, 5 g/l NaCl and 18 g/l Bacto-agar, which had been supplemented with 25 mg/l kanamycin. Incubation was carried out for 2 days at 33° C.

Plasmid DNA was isolated from a transformant by conventional methods (Peters-Wendisch et al., 1998, Microbiology 144, 915-927) and checked by restriction cleavage. The resulting strain was called ATCC13032pCREmetF.

### Example 4

### Preparation of methionine with the strain C. glutamicum ATCC13032/pCREmetF

The C. glutamicum strain ATCC13032/pCREmetF obtained in example 3 was cultured in a nutrient medium suitable for the production of methionine and the methionine content in the culture supernatant was determined.

For this, the strain was first incubated on an agar plate with the corresponding antibiotic (brain-heart agar with kanamycin (25 mg/1)) for 24 hours at 33° C. Starting from this agar plate culture, a preculture was seeded (10 ml medium in a 100 ml conical flask). The medium MM was used as the medium for the preculture.

Medium MM

| | | |
|---|---|---|
| CSL (corn steep liquor) | 5 | g/l |
| MOPS (morpholinopropanesulfonic acid) | 20 | g/l |
| Glucose (autoclaved separately) | 50 | g/l |
| Salts: | | |
| (NH₄)₂SO₄ | 25 | g/l |
| KH₂PO₄ | 0.1 | g/l |
| MgSO₄*7H₂O | 1.0 | g/l |
| CaCl₂*2H₂O | 10 | mg/l |
| FeSO₄*7H₂O | 10 | mg/l |
| MnSO₄*H₂O | 5.0 | mg/l |
| Biotin (sterile-filtered) | 0.01 | mg/l |
| Vitamin B12 (sterile-filtered) | 0.02 | mg/l |
| Thiamine*HCl (sterile-filtered) | 0.2 | mg/l |
| CaCO₃ | 25 | g/l |

The CSL, MOPS and the salt solution were brought to pH 7 with aqueous ammonia and autoclaved. The sterile substrate and vitamin solutions were then added, as well as the CaCO₃ autoclaved in the dry state.

Kanamycin (25 mg/l) was added to this. The preculture was incubated for 16 hours at 33° C. at 240 rpm on a shaking machine. A main culture was seeded from this preculture such that the initial OD (660 nm) of the main culture was 0.1. Medium MM was also used for the main culture.

Culturing is carried out in a 10 ml volume in a 100 ml conical flask with baffles. Kanamycin (25 mg/l) was added. Culturing was carried out at 33° C. and 80% atmospheric humidity.

After 72 hours, the OD was determined at a measurement wavelength of 660 nm with a Biomek 1000 (Beckmann Instruments GmbH, Munich). The amount of methionine formed was determined with an amino acid analyzer from Eppendorf-BioTronik (Hamburg, Germany) by ion exchange chromatography and post-column derivation with ninhydrin detection.

The result of the experiment is shown in Table 1.

TABLE 1

| | OD | Methionine |
|---|---|---|
| Strain | (660 nm) | mg/l |
| ATCC13032 | 10.3 | 1.4 |
| ATCC13032/pCREmetF | 11.2 | 7.3 |

### BRIEF DESCRIPTION OF THE FIGURE:

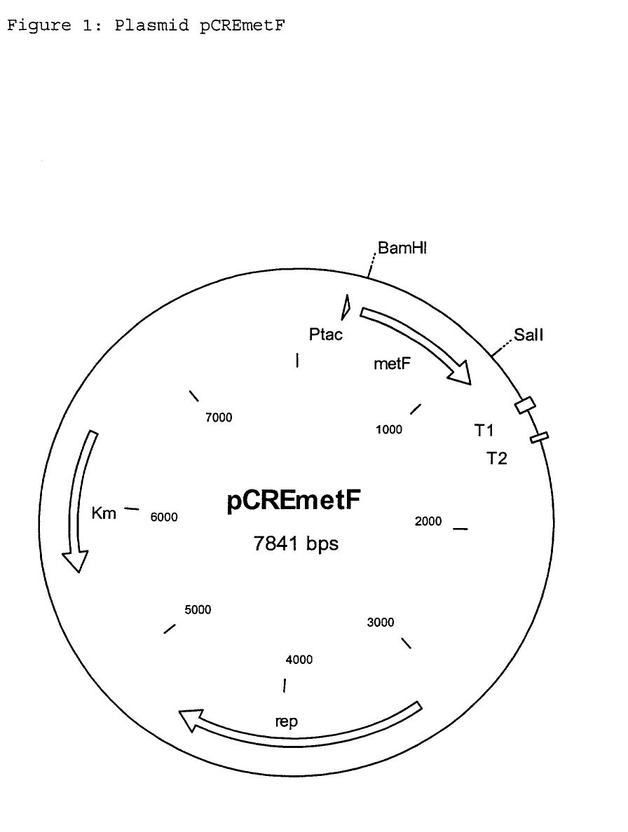

FIG. 1: Plasmid pCREmetF

The abbreviations used have the following meaning:

| | |
|---|---|
| Km: | Resistance gene for kanamycin |
| metF: | metF gene of C. glutamicum |
| Ptac: | tac promoter |
| T1 T2: | Terminator T1T2 of the rrnB gene of E. coli |
| rep: | Plasmid-coded replication origin for C. glutamicum |
| | (of pHM1519) |
| BamHI: | Cleavage site of the restriction enzyme BamHI |
| SalI: | Cleavage site of the restriction enzyme SalI |

This disclosure is based on priority documents DE 100 53 942.4, DE 101 09 686.0 and US 60/294,279, each incorporated by reference.

Obviously, numerous modifications of the invention are possible in view of the above teachings. Therefore, within the scope of the appended claims, the invention may be practiced otherwise than as specifically described herein.

1. An isolated polynucleotide, comprising a polynucleotide sequence selected from the group consisting of
a) polynucleotide which is at least 70% identical to a polynucleotide that codes for a polypeptide which comprises the amino acid sequence of SEQ ID No. 2,
b) polynucleotide which codes for a polypeptide that comprises an amino acid sequence which is at least 70% identical to the amino acid sequence of SEQ ID No. 2,
c) polynucleotide which is complementary to the polynucleotides of a) or b), and
d) polynucleotide comprising at least 15 successive nucleotides of the polynucleotide sequence of a), b) or c).

2. The polynucleotide as claimed in claim 1, which is capable of replication in coryneform bacteria.

3. The polynucleotide as claimed in claim 1, wherein the polynucleotide is an RNA.

4. The polynucleotide as claimed in claim 2, comprising the nucleic acid sequence as shown in SEQ ID No. 1.

5. The DNA as claimed in claim 2 which is capable of replication, comprising
(i) the nucleotide sequence shown in SEQ ID No. 1, or
(ii) at least one sequence which corresponds to sequence (i) within the range of the degeneration of the genetic code, or
(iii) at least one sequence which hybridizes with the sequence complementary to sequence (i) or (ii), and optionally
(iv) sense mutations of neutral function in (i).

6. The polynucleotide sequence as claimed in claim 2, which codes for a polypeptide which comprises the amino acid sequence in SEQ ID No. 2.

7. A coryneform bacterium in which the metF gene is enhanced.

8. A coryneform bacterium serving as a host cell, that contains a vector which carries a polynucleotide as claimed in claim 1.

9. A process for the fermentative preparation of L-amino acids, comprising:
a) fermentation of the coryneform bacteria which produce the desired L-amino acid and in which at least the metF gene or nucleotide sequences which code for it are enhanced;
b) concentration of the L-amino acid in the medium or in the cells of the bacteria, and
c) isolation of the L-amino acid.

10. The process as claimed in claim 9, wherein bacteria in which further genes of the biosynthesis pathway of the desired L-amino acid are additionally enhanced are employed.

11. The process as claimed in claim 9, wherein bacteria in which the metabolic pathways which reduce the formation of the desired L-amino acid are at least partly eliminated are employed.

12. The process as claimed in claim 9, wherein a strain transformed with a plasmid vector is employed, and the plasmid vector carries the nucleotide sequence which codes for the metF gene.

13. The process as claimed in claim 9, wherein the expression of the polynucleotide(s) which code(s) for the metF gene is enhanced, in particular over-expressed.

14. The process as claimed in claim 9, wherein the catalytic properties of the enzyme encoded by metF are increased.

15. The process as claimed in claim 9, wherein for the preparation of L-methionine, coryneform microorganisms have one or more enhanced genes selected from the group consisting of
15.1 the lysC gene which codes for a feed back resistant aspartate kinase,
15.2 the gap gene which codes for glycerolaldehyde 3-phosphate dehydrogenase,
15.3 the pgk gene which codes for 3-phosphoglycerate kinase,
15.4 the pyc gene which codes for pyruvate carboxylase,
15.5 the tpi gene which codes for triose phosphate isomerase,
15.6 the metA gene which codes for homoserine O-acetyltransferase,
15.7 the metB gene which codes for cystathionine gamma-synthase,
15.8 the aecD gene which codes for cystathionine gamma-lyase,
15.9 the glyA gene which codes for serine hydroxymethyltransferase,
15.10 the metY gene which codes for O-acetylhomoserine sulfhydrylase.

16. The process as claimed in claim 9, wherein for the preparation of L-methionine, the coryneform microorganisms have one or more attenuated genes selected from the group consisting of
16.1 the thrB gene which codes for homoserine kinase,
16.2 the ilvA gene which codes for threonine dehydratase,
16.3 the thrc gene which codes for threonine synthase,
16.4 the ddh gene which codes for meso-diaminopimelate D-dehydrogenase,
16.5 the pck gene which codes for phosphoenol pyruvate carboxykinase,
16.6 the pgi gene which codes for glucose 6-phosphate isomerase,
16.7 the poxB gene which codes for pyruvate oxidase.

17. The process of claims 9, wherein microorganisms of the species Corynebacterium glutamicum are employed.

18. The process as claimed in claim 17, wherein the Corynebacterium glutamicum strain ATCC13032/pCREmetF is employed.

19. A process for preparing an L-methionine-containing animal feedstuffs additive, comprising:
a) culture and fermentation of an L-methionine-producing microorganism in a fermentation medium;
b) removal of water from the L-methionine-containing fermentation broth (concentration);
c) removal of an amount of 0 to 100 wt. % of the biomass formed during the fermentation; and
d) drying of the fermentation broth obtained according to b) and/or c) to obtain the animal feedstuffs additive in the desired powder or granule form.

20. The process as claimed in claim 19, wherein microorganisms are employed in which further genes of the biosynthesis pathway of L-methionine are additionally enhanced.

21. The process as claimed in claim 20, wherein microorganisms are employed in which the metabolic pathways which reduce the formation of L-methionine are at least partly eliminated.

22. The process as claimed in claim 20, wherein expression of the polynucleotide(s) which code(s) for the metF gene is enhanced.

23. The process of claim 19, wherein microorganisms of the species Corynebacterium glutamicum are employed.

24. The process as claimed in claim 23, wherein the Corynebacterium glutamicum strain ATCC13032/pCREmetF is employed.

25. The process as claimed in claimed claim 19, wherein one or more of the following steps are additionally carried out:
e) addition of one or more organic substances, including L-methionine and/or D-methionine and/or the racemic mixture D,L-methionine, to the products obtained according to b), c) and/or d);
f) addition of auxiliary substances selected from the group consisting of silicas, silicates, stearates, grits and bran to the substances obtained according to b) to e) for stabilization and to increase storability; or
g) conversion of the substances obtained according to b) to f) into a form stable in rumen, by coating them with film-forming agents.

26. The process as claimed in claim 19 or 25, wherein a portion of the biomass is removed.

27. A process as claimed in claim 26, wherein essentially 100% of the biomass is removed.

28. The process as claimed in claim 19 or 25, wherein the water content is up to 5 wt. %.

29. The process as claimed in claim 28, wherein the water content is less than 2 wt. %.

30. The process as claimed in claim 25, wherein the film-forming agents are metal carbonates, silicas, silicates, alginates, stearates, starches, gums or cellulose ethers.

31. An animal feedstuffs additive prepared as claimed in claim 19.

32. An animal feedstuffs additive as claimed in claim 31, which comprises 1 wt. % to 80 wt. % L-methionine, D-methionine, D,L-methionine or a mixture thereof, based on the dry weight of the animal feedstuffs additive.

33. A process for obtaining RNA, cDNA or DNA in order to isolate nucleic acids, or polynucleotides or genes which code for methylene tetrahydrofolate reductase or have a high similarity to the sequence of the methylene tetrahydrofolate reductase gene, which comprises employing the polynucleotide sequences as claimed in claim 1 as hybridization probes.


SEQUENCE LISTING (4 sequences)

SEQ ID NO: 1 - DNA, 1551 bp, Corynebacterium glutamicum
  CDS (299)..(1345)
gcgtcaaggacggactcaagtttttcagaagaattcttatggccttgcgccgccaggaaaccagcccacgcataaagaggacggattcgctttcctccattgagcacgaaactgcgaagatgggccacagcatctgtgacaggagcgccgatatcagcaattgttagctcttgagcatcgaggaactgcgtcaaacgatctcgcacgacctccggaaatttgtcgaggtcaaggtcatgggcatcgaaactgctcaaggagacgtccttcaatcgaatagggggatgcgggctgaattttggtggaggtgaataaatgccagaggcagtcccaacaaaacactctcatcacactaagatacccaggcatgtccctaacgaacatcccagcctcatctcaatgggcaattagcgacgttttgaagcgtccttcacccggccgagtacctttttctgtcgagtttatgccaccccgcgacgatgcagctgaagagcgtctttaccgcgcagcagaggtcttccatgacctcggtgcatcgtttgtctccgtgacttatggtgctggcggatcaacccgtgagagaacctcacgtattgctcgacgattagcgaaacaaccgttgaccactctggtgcacctgaccctggttaaccacactcgcgaagagatgaaggcaattcttcgggaatacctagagctgggattaacaaacctgttggcgcttcgaggagatccgcctggagacccattaggcgattgggtgagcaccgatggaggactgaactatgcctctgagctcatcgatcttattaagtccactcctgagttccgggaattcgacctcggtatcgcctccttccccgaagggcatttccgggcgaaaactctagaagaagacaccaaatacactctggcgaagctgcgtggaggggcagagtactccatcacgcagatgttctttgatgtggaagactacctgcgacttcgtgatcgccttgtcgctgcagaccccattcatggtgcgaagccaatcattcctggcatcatgcccattaccgagctgcggtctgtgcgtcgacaggtcgaactctctggtgctcaattgccgagccaactagaagaatcacttgttcgagctgcaaacggcaatgaagaagcgaacaaagacgagatccgcaaggtgggcattgaatattccaccaatatggcagagcgactcattgccgaaggtgcggaagatctgcacttcatgacgcttaacttcacccgtgcaacccaagaagtgttgtacaaccttggcatggcgcctgcttggggagcagagcacggccaagacgcggtgcgttaagccctcttaggaatcatgaagggggagggcggtgatcaatacggcaaacggccgttgatccccgtcaaacctaaactgcctgagcaagtcagtgaagccgagagagcgatacaggctaaacgcatggttcgcctcatcgtcgacctcgggtgtagacaaaatggcaaaagtgttttgtttgtcttttaacagttcatgcatca

SEQ ID NO: 2 - protein, 349 aa, Corynebacterium glutamicum
LVEVNKCQRQSQQNTLITLRYPGMSLTNIPASSQWAISDVLKRPSPGRVPFSVEFMPPRDDAAEERLYRAAEVFHDLGASFVSVTYGAGGSTRERTSRIARRLAKQPLTTLVHLTLVNHTREEMKAILREYLELGLTNLLALRGDPPGDPLGDWVSTDGGLNYASELIDLIKSTPEFREFDLGIASFPEGHFRAKTLEEDTKYTLAKLRGGAEYSITQMFFDVEDYLRLRDRLVAADPIHGAKPIIPGIMPITELRSVRRQVELSGAQLPSQLEESLVRAANGNEEANKDEIRKVGIEYSTNMAERLIAEGAEDLHFMTLNFTRATQEVLYNLGMAPAWGAEHGQDAVR

SEQ ID NO: 3 - DNA, 46 bp, Artificial sequence
  Synthetic DNA
gatctaggatccaaaggaggacaaccatgtccctaacgaacatccc

SEQ ID NO: 4 - DNA, 30 bp, Artificial sequence
  Synthetic DNA
gatctactcgagttcttctagttggctcgg